(19) United States
(12) Patent Application Publication
(10) Pub. No.: US 2007/0191304 A1
(43) Pub. Date: Aug. 16, 2007

(54) Methods for performing percutaneous coronary intervention

(76) Inventors: Gilles Montalescot, Paris (FR); Flavia Dietrich Neto, Basking Ridge, NJ (US); Luis Toro-Figueroa, Princeton, NJ (US); Philippe Gabriel Steg, Paris (FR); Marie-France Bregeault, Brunoy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/514,366

(22) Filed: Sep. 1, 2006

Related U.S. Application Data
(60) Provisional application No. 60/713,329, filed on Sep. 2, 2005.

(51) Int. Cl. A61K 31/727

(52) U.S. Cl. 514/56

(57) ABSTRACT

Methods for performing percutaneous coronary intervention in a patient in need thereof comprising administering intravenously a bolus comprising an effective amount of enoxaparin sodium to the patient after sheath insertion and prior to the percutaneous coronary intervention are described. Also described are methods for preventing or treating thrombosis, such as thrombotic episodes, in a human percutaneous coronary intervention patient by treating that patient with enoxaparin.

This application claims priority under 35 U.S.C. § 119(e) to Provisional Application No. 60/713,329, filed Sep. 2, 2005, the entire contents of which are hereby incorporated herein by reference.

This invention relates to methods for performing percutaneous coronary intervention in a patient in need thereof comprising administering intravenously a bolus comprising an effective amount of enoxaparin sodium (sometimes referred to herein as “enoxaparin”) to the patient after sheath insertion and prior to the percutaneous coronary intervention (“PCI”). The invention also relates to methods for preventing or treating thrombosis, such as thrombotic episodes, in a human percutaneous coronary intervention patient by treating that patient with enoxaparin.

Enoxaparin sodium is available from sanofi-aventis under the trademark Lovenox® (Clexane® in some other countries). Known enoxaparin sodium dosage regimens include those shown below:

Dosage Regimen

| | | Severe Renal |
|---|---|---|
| Indication | Standard Regimen | Impairment |
| Prophylaxis in abdominal surgery | 40 mg SC once daily | 30 mg SC once daily |
| Prophylaxis in knee replacement | 30 mg SC every 12 | 30 mg SC once daily |
| surgery | hours | |
| Prophylaxis in hip replacement | 30 mg SC every 12 | 30 mg SC once daily |
| surgery | hours or 40 mg SC | |
| | once daily | |
| Prophylaxis in medical patients | 40 mg SC once daily | 30 mg SC once daily |
| Inpatient treatment of acute DVT with | 1 mg/kg SC every | 1 mg/kg SC once |
| or without pulmonary embolism | 12 hours (with | daily |
| | warfarin) | |
| Outpatient treatment of acute DVT | 1.5 mg/kg SC once | 1 mg/kg SC once |
| without pulmonary embolism | daily (with warfarin) | daily |
| Prophylaxis of ischemic | 1 mg/kg SC every | 1 mg/kg SC once |
| complications of unstable angina and | 12 hours (with | daily |
| non-Q-wave MI | aspirin) | |

Elderly: For treatment of acute MI in elderly patients >=75 years of age, initiate dosing with 0.75 mg/kg SC every 12 hours

Provided herein are methods for performing percutaneous coronary intervention in a patient in need thereof. The methods comprise administering intravenously a bolus comprising an effective amount of enoxaparin to the patient after sheath insertion and prior to the percutaneous coronary intervention.

Also provided are methods for preventing thrombosis in a human patient in need thereof. The methods comprise administering intravenously a bolus comprising an effective amount of enoxaparin to the patient after sheath insertion and prior to the percutaneous coronary intervention.

Also provided are methods for treating thrombosis in a human patient in need thereof. The methods comprise administering intravenously a bolus comprising an effective amount of enoxaparin to the patient after sheath insertion and prior to the percutaneous coronary intervention.

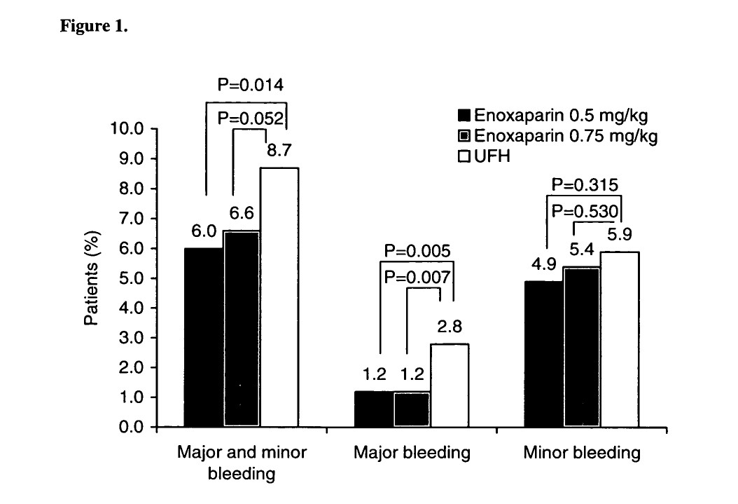

FIG. 1 shows non-CABG-related major and minor bleeding at 48 hours in the STEEPLE study. P values are comparison of enoxaparin groups with the UFH group. 95% confidence interval for adjusted differences between groups for non-CABG major and minor bleeding at 48 hours: enoxaparin 0.5 mg/kg and UFH, (−4.8 to −0.5%), enoxaparin 0.75 mg/kg and UFH, (−4.1% to 0.0%); noninferiority margin of 2.6%.

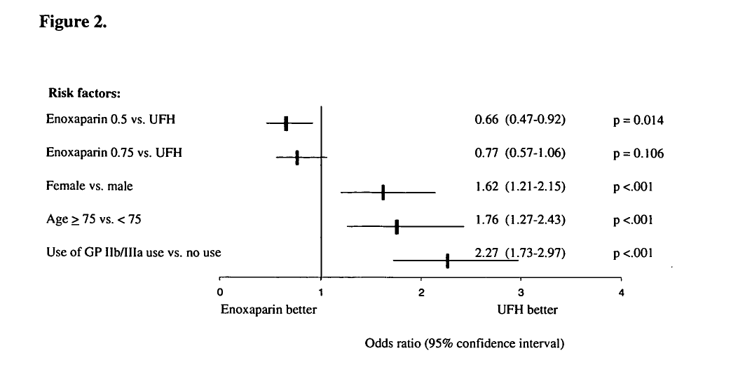

FIG. 2 shows the odds ratio and 95% confidence interval of risk factors for non-CABG related major and minor bleeding at 48 hours in the STEEPLE study (multivariate analysis).

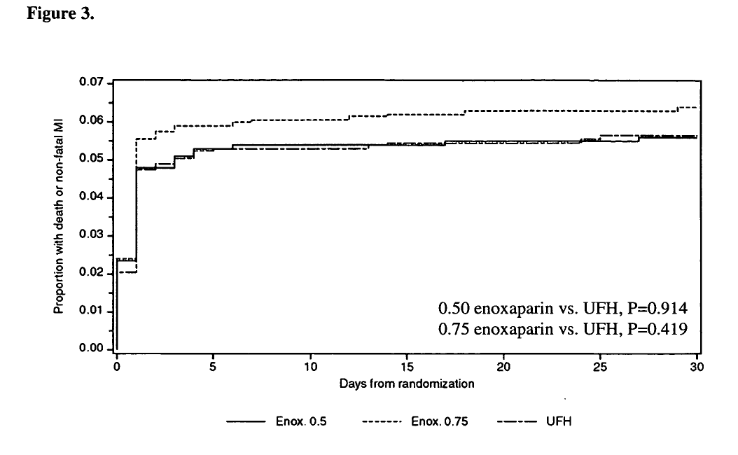

FIG. 3 shows Kaplan Meier curve for all cause mortality and nonfatal myocardial infarction at 30 days in the STEEPLE study.

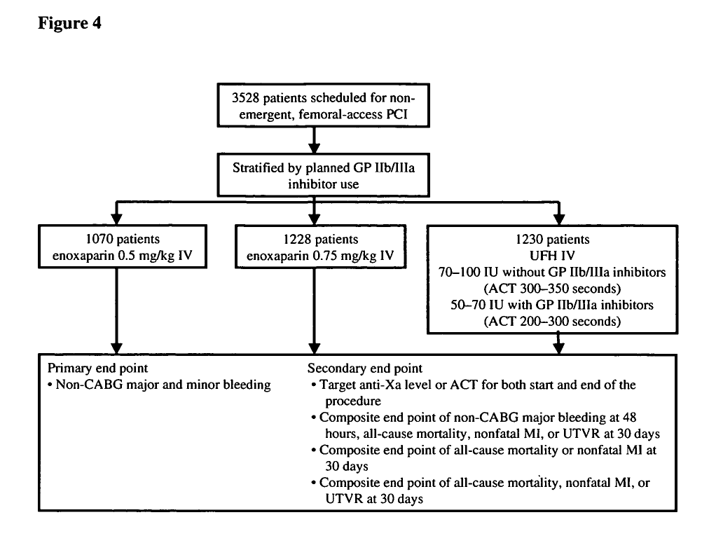

FIG. 4 shows the STEEPLE study design. ACT, activated clotting time; CABG, coronary artery bypass graft; GP, glycoprotein; IV, intravenous; MI, myocardial infarction; PCI, percutaneous coronary intervention; UTVR, urgent target vessel revascularization.

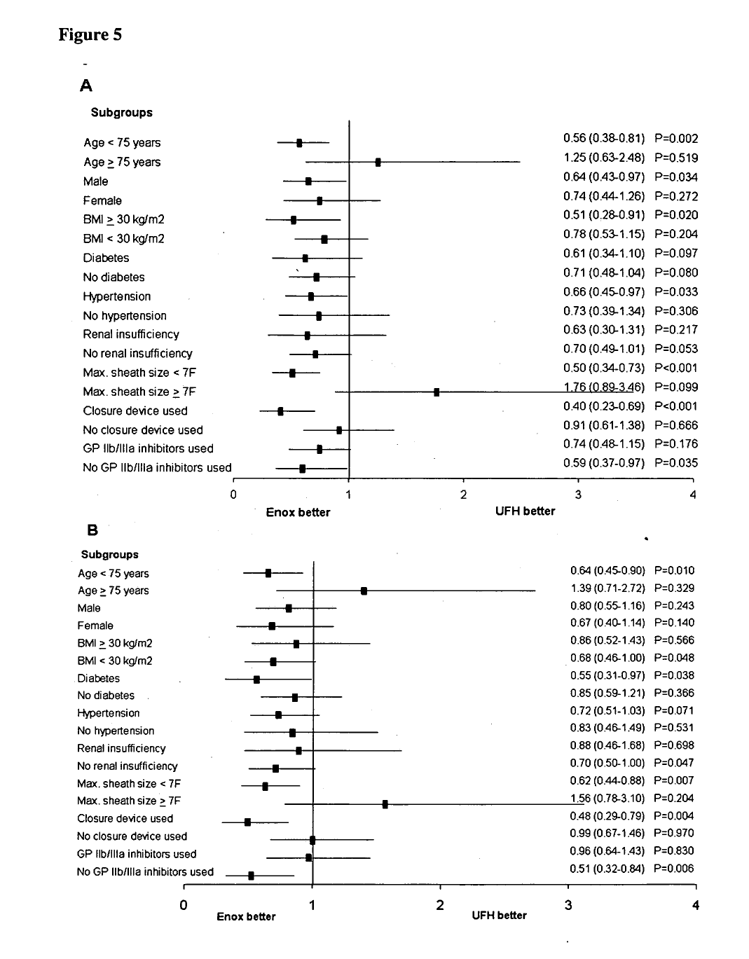

FIG. 5 shows a subgroup analysis for the STEEPLE study of non-CABG-related major or minor bleeding at 48 hours, comparison of enoxaparin 0.5 mg/kg (A) and 0.75 mg/kg (B) with UFH.

As used herein, “enoxaparin sodium” refers to the low molecular weight heparin (LMWH) approved by the U.S. Food and Drug Administration (FDA), or any other regulatory agency outside of the United States, as Lovenox® (enoxaparin sodium injection), Clexane® or Klexane®, and any LMWH approved by the FDA, or any other regulatory agency outside of the United States, pursuant to an application citing Lovenox® (enoxaparin sodium injection), Clexane® or Klexane® as the listed drug. Enoxaparin sodium is available from sanofi-aventis and sold in the United States in the form of enoxaparin sodium injection, under the trademark Lovenox® (Clexane® in some other countries). In general, enoxaparin sodium is obtained by alkaline degradation of heparin benzyl ester derived from porcine intestinal mucosa. Its structure is characterized, for example, by a 2-0-sulfo-4-enepyranosuronic acid group at the non-reducing end and a 2-N,6-0-disulfo-D-glucosamine at the reducing end of the chain. The average molecular weight is about 4500 daltons. The molecular weight distribution is:

| | | |
|---|---|---|
| <2000 | daltons | ≦20% |
| 2000 to 8000 | daltons | ≧68% |
| >8000 | daltons | ≦18% |

Enoxaparin sodium injection is a sterile aqueous solution containing enoxaparin sodium. Enoxaparin sodium injection is available from sanofi-aventis at 100 mg/ml in prefilled syringes (30 mg/0.3 mL pre-filled syringes, 40 mg/0.4 mL pre-filled syringes, 60 mg/0.6 mL pre-filled syringes, 80 mg/0.8 mL pre-filled syringes, and 100 mg/1.0 mL pre-filled syringes), graduated prefilled syringes, multiple-dose vials (300 mg/3.0 mL multi-dose vials), and ampoules (30 mg/0.3 mL). Enoxaparin sodium injection 100 mg/mL concentration contains 10 mg enoxaparin sodium (approximate anti-Factor Xa activity of 1000 IU [with reference to the W.H.O. First International Low Molecular Weight Heparin Reference Standard]) per 0.1 mL water for injection. Enoxaparin sodium injection is also available from sanofi-aventis at 150 mg/ml in graduated prefilled syringes (90 mg/0.6 mL pre-filled syringes, 120 mg/0.8 mL pre-filled syringes, and 150 mg/1.0 mL pre-filled syringes). Enoxaparin sodium injection 150 mg/mL concentration contains 15 mg enoxaparin sodium (approximate anti-Factor Xa activity of 1500 IU [with reference to the W.H.O. First International Low Molecular Weight Heparin Reference Standard]) per 0.1 mL water for injection.

The enoxaparin sodium injection prefilled syringes and graduated prefilled syringes are preservative-free and intended for use only as a single-dose injection. There are also multiple-dose vials and those contain 15 mg/1.0 mL benzyl alcohol as a preservative. The pH of the injection is 5.5 to 7.5. Enoxaparin sodium injection may also be administered in an arterial line for a hemodialysis indication.

As used herein, reference to administration of enoxaparin sodium once daily means, for example, administration every twenty-four hours plus or minus four hours.

The term “prevent,” “preventing” and “prevention” refers to the administration of therapy an individual who may ultimately manifest at least one symptom of a disease or condition (e.g., thrombosis) but who has not yet done so, to reduce the chance that the individual will develop the symptom of a disease or condition over a given period of time. Such a reduction may be reflected, for example, in a delayed onset of the at least one symptom of a disease or condition in the patient.

As used herein, the term “treat,” “treating” or “treatment” refers to the administration of therapy to an individual who already manifests at least one symptom of a disease or condition (e.g., thrombosis), or who has previously manifested at least one symptom of a disease or condition.

“Body weight” refers to the weight of a patient that is determined by actual weighing or by estimation prior to administration of enoxaparin sodium. In the event that the body weight of a patient is estimated prior to administration of the first dose of enoxaparin sodium, the body weight of the patient may be subsequently determined by actual weighing before any subsequent dose of enoxaparin, and the amount of enoxaparin administered to the patient with the next subsequent dose adjusted accordingly.

“Kg” refers to body weight of the patient in kilograms.

“Percutaneous coronary intervention” (PCI) encompasses a variety of procedures used to treat patients with diseased arteries of the heart, for example, chest pain caused by a build-up of fats, cholesterol, and other substances from the blood (referred to as plaque) that can reduce blood flow to a near trickle, or a heart attack caused by a large blood clot that completely blocks the artery. Typically, PCI is performed by threading a slender balloon-tipped tube—a catheter—from an artery in the groin to a trouble spot in an artery of the heart (this is referred to as percutaneous transluminal coronary angioplasty—also known as PTCA, coronary artery balloon dilation or balloon angioplasty). The balloon is then inflated, compressing the plaque and dilating (widening) the narrowed coronary artery so that blood can flow more easily. This is often accompanied by inserting an expandable metal stent. Stents are wire mesh tubes used to prop open arteries after PTCA.

A “sheath” is a tube or other device which is inserted into an artery and used to guide a catheter into place.

Provided is a method for performing percutaneous coronary intervention in a patient in need thereof comprising administering intravenously a bolus comprising an effective amount of enoxaparin to the patient after sheath insertion and prior to the percutaneous coronary intervention. Also provided is a method for preventing or treating thrombosis, such as thrombotic episodes, in a human percutaneous coronary intervention patient by treating that patient with enoxaparin.

In certain embodiments, the enoxaparin is administered immediately prior to the percutaneous coronary intervention.

In certain embodiments, sheath removal occurs immediately after the percutaneous coronary intervention. In certain embodiments, sheath removal occurs 4 to 6 hours after the percutaneous coronary intervention.

In certain embodiments, enoxaparin is administered in an amount such that the patient achieves an anti-Xa level of 0.5 to 1.8 IU/mL

In certain embodiments, enoxaparin is administered in an amount such that the patient achieves an anti-Xa level of 0.5 to 1.2 IU/mL.

In certain embodiments, enoxaparin is administered in an amount sufficient to reduce the primary end-point of any bleeding as compared with an ACT-adjusted UFH regimen.

In certain embodiments, enoxaparin is administered at a dosage of 0.5 mg/kg. In certain embodiments, enoxaparin is administered at a dosage of 0.75 mg/kg.

Administration of enoxaparin sodium is, for example, by intravenous injection directly with a syringe, and further for example, by injection into a pre-established i.v. line. A bolus dose of enoxaparin may be administered, for example, in about 10 seconds or less, or in from about 10 to about 20 seconds, or from about 15 to about 30 seconds, or from about 30 seconds to about 1 minute, or from about 1 to about 5 minutes, or from about 5 to about 10 minutes, or from about 10 to about 30 minutes.

In certain embodiments, the patient exhibits a significant reduction in major bleeding as compared with a subject who had been administered UFH prior to undergoing percutaneous coronary intervention. In certain embodiments, the significant reduction in major bleeding is at least 25%. In certain embodiments, the significant reduction in major bleeding is at least 40%. In certain embodiments, the significant reduction in major bleeding is at least 55%.

In certain embodiments, the patient achieves target anticoagulation level at a rate significantly increased as compared to the rate for a subject receiving UFH. In certain embodiments, the patient achieves target anticoagulation level at a rate at least 2-fold faster than the rate for a subject receiving UFH. In certain embodiments, the patient achieves target anticoagulation level at a rate at least 3-fold faster than the rate for a subject receiving UFH. In certain embodiments, the patient achieves target anticoagulation level at a rate 4-fold faster than the rate for a subject receiving UFH.

In certain embodiments, the patient is administered a second bolus of enoxaparin during the percutaneous coronary intervention. In certain embodiments, the amount of enoxaparin in the second bolus is less than the amount of enoxaparin initially administered. In certain embodiments, the amount of enoxaparin in the second bolus is about half of the amount of enoxaparin initially administered.

In certain embodiments, the patient is administered at least one additional therapeutic agent. Such “co-therapy” (or “combination-therapy”) may include the following examples:

administration of each agent in a sequential manner in a regimen to provide beneficial effects of the drug combination; and/or

co-administration of the aforementioned components in a substantially simultaneous manner (e.g., as in a single injection having a fixed ratio of these active agents or in multiple, separate injections for each agent, etc.).

Thus, the methods described herein are not limited in the sequence of administration; the enoxaparin sodium may be administered either prior to, at the same time with or after administration of the other agent. In addition, enoxaparin sodium may be administered alone or in combination with other treatments, e.g., drug-eluting stent.

In certain embodiments, the at least one additional therapeutic agent is chosen from aspirin and thienopyridines. In certain embodiments, the at least one additional therapeutic agent is chosen from clopidogrel and GP IIb/IIIa inhibitors. In certain embodiments, the at least one additional therapeutic agent is chosen from GP IIb/IIIa inhibitors. GP IIb/IIIa inhibitors include abciximab (marketed as ReoPro®), tirofiban (marketed as Aggrastat®), and eptifibatide (marketed as Integrelin®).

In certain embodiments, enoxaparin is administered in about the same amount as when the GP IIb/IIIa inhibitor is not administered.

Thus, provided are methods for combination therapy for treatment of a human PCI patient, which comprises administering to the patient

intravenously a bolus comprising an effective amount of enoxaparin to the patient after sheath insertion and prior to the procedure; and

at least one additional therapeutic agent.

Also provided are methods for preventing or treating thrombosis in a human PCI patient, which comprises administering to the patient

intravenously a bolus comprising an effective amount of enoxaparin to the patient after sheath insertion and prior to the procedure; and

at least one additional therapeutic agent.

Also provided are methods for performing percutaneous coronary intervention in a patient in need thereof comprising

administering to the patient intravenously a bolus comprising an effective amount of enoxaparin to the patient after sheath insertion and prior to the procedure; and

implanting into a blood vessel of the patient a drug-eluting stent.

In certain embodiments, prevention of thrombosis comprises prevention of thrombotic episodes. In certain embodiments, treatment of thrombosis comprises treatment of thrombotic episodes.

In providing enoxaparin sodium dosage regimens, the dosage regimen for administration of enoxaparin sodium is based on the particular indication. For each indication, methods are provided herein that include methods for preventing and/or treating thrombotic episodes in a human PCI patient.

It will be readily apparent to one of ordinary skill in the relevant arts that other suitable modifications and adaptations to the methods and applications described herein are suitable and may be made without departing from the scope of the invention or any embodiment thereof. Having now described the present invention in detail, the same will be more clearly understood by reference to the following examples of the invention, which are included herewith for purposes of illustration only and are not intended to be limiting of the invention.

### EXAMPLE 1

### Patient Treatment

A patient is identified as a candidate for PCI. After sheath insertion but prior to PCI, the patient is administered a dosage of 0.5 mg of enoxaparin sodium per kg of the patient's body weight by intravenous bolus over a period of approximately 20 seconds. The anti-Xa level experienced by the patients is analyzed centrally and the patient is determined to have achieved an anti-Xa level of 1 IU/mL.

### EXAMPLE 2

### Patient Treatment

A patient is identified as a candidate for PCI. After sheath insertion but prior to PCI, the patient is administered a dosage of 0.75 mg of enoxaparin sodium per kg of the patient's body weight by intravenous bolus over a period of approximately 20 seconds. The anti-Xa level experienced by the patients is analyzed centrally and the patient is determined to have achieved an anti-Xa level of 1 IU/mL.

### EXAMPLE 3

### The STEEPLE Study

The American College of Cardiology/American Heart Association (ACC/AHA) and the European Society of Cardiology (ESC) guidelines recommend the use of activated clotting time (ACT)-adjusted intravenous unfractionated heparin (UFH) during PCI. However, there is interest in improving anticoagulation regimens during PCI, especially given the limitations of UFH such as its unpredictable effect on coagulation, the need for repeated coagulation monitoring, the narrow therapeutic window, the inducement of platelet activation, and the risk of thrombocytopenia.

Low-molecular-weight heparins (LMWHs), such as enoxaparin, are being used more frequently as anticoagulants during PCI in acute coronary syndrome (ACS) patients and also during elective procedures. They have a more stable and predictable anticoagulant dose-response, obviating the necessity for coagulation monitoring; a longer half-life; and a greater anti-factor Xa:IIa activity, which results in less thrombin generation and activation. LMWHs are associated with reduced platelet activation, von Willebrand factor release, and inflammation.

Small and/or noncomparative trials have shown the feasibility of a single intravenous bolus of enoxaparin 1 mg/kg, 0.75 mg/kg, and 0.5 mg/kg in patients undergoing PCI with or without the administration of glycoprotein (GP) IIb/IIIa inhibitors. However, these uncontrolled studies did not allow definite conclusions in comparison with standard anticoagulation with UFH, and the optimum intravenous bolus dose of enoxaparin during PCI remains to be established. A meta-analysis of the data from randomized studies comparing the use of intravenous LMWH and intravenous UFH in patients undergoing PCI found no difference in the occurrence of ischemic events and a nonsignificant trend toward a reduction in major bleeding with LMWH.

Methods

Patients

STEEPLE is a prospective, randomized, open-label, parallel-group trial evaluating the safety and efficacy of enoxaparin 0.5 mg/kg and 0.75 mg/kg intravenously versus an ACT-adjusted, intravenous UFH regimen in patients undergoing non-emergent PCI. Between January 2004 and December 2004, 3528 patients were randomized across 124 sites in 9 countries in Australasia, Europe, and North America.

Patients were eligible to enter the study if they were scheduled to have single- or multi-vessel non-emergent PCI with a femoral approach and did not meet any of the exclusion criteria including recent thrombolysis, planned staged procedure, increased bleeding risk, treatment with a parenteral antithrombotic prior to the procedure, or a known hypersensitivity to study drugs. This study was conducted according to the Declaration of Helsinki (Tokyo, 2004) and in keeping with local regulations. All patients gave informed consent and approval for the study was obtained through institutional review boards at each site.

Study Protocol

Eligible patients were randomized to receive an intravenous bolus of ACT-adjusted UFH according to the current guidelines or enoxaparin 0.5 mg/kg or 0.75 mg/kg intravenously without coagulation monitoring. Patients were randomized in a 1:1:1 ratio and stratified according to planned GP IIb/IIIa inhibitor use. All patients received aspirin (75 to 500 mg/day) and thienopyridines according to local practice. The use of all concomitant medications was consistent with local standards of care.

Patients randomized to either enoxaparin group received a single intravenous bolus of enoxaparin after sheath insertion and immediately prior to the procedure. The randomized dosage was used regardless of whether the investigator had chosen to initiate concomitant GP IIb/IIIa inhibitor treatment. Patients randomized to the UFH group who were not receiving concurrent GP IIb/IIIa inhibitor treatment were given an initial bolus of UFH 70 to 100 IU/kg, based on the operator's standard practice, to achieve a target ACT of 300 to 350 seconds. Similarly, patients who received concurrent GP IIb/IIIa inhibitor treatment were given an initial intravenous bolus of 50 to 70 lU/kg to achieve a target ACT of 200 to 300 seconds. Additional boluses of UFH before the start of PCI were given only to patients who did not reach the lower limit of the ACT target. UFH was re-administered during the procedure when ACT measurements dropped below the recommended range. In all centers, ACT was measured with a standardized Hemochron device. When procedures were prolonged by more than 2 hours in patients allocated to enoxaparin, an additional bolus (half of the original dose) was recommended. Closure devices were allowed as per local practice. Sheath removal was authorized with the ACT between 150 and 180 seconds in the UFH group, 4 to 6 hours after the end of PCI in the enoxaparin 0.75 mg/kg group and immediately after the end of PCI in the enoxaparin 0.5 mg/kg group. Importantly, no monitoring or verification of anticoagulation was required before sheath removal in the patients receiving enoxaparin.

End Points

The primary end point of the trial was the incidence of non-coronary artery bypass graft (non-CABG)-related major and minor bleeding at 48 hours after the index PCI. Major bleeding was defined as a bleeding event that resulted in death; was retroperitoneal, intracranial, or intraocular; resulted in hemodynamic compromise requiring specific treatment; required intervention (surgical or endoscopic) or decompression of a closed space to stop or control the event; was clinically overt with transfusion of at least 1 unit of packed red blood cells or whole blood; or was clinically overt with a decrease in hemoglobin of ≧3 g/dL (or a decrease in hematocrit of ≧10%). Minor bleeding was defined as all bleeding events not meeting the criteria for major bleeding, but with at least one of the following features: gross hematuria not associated with trauma; epistaxis that was prolonged or requiring intervention; gastrointestinal hemorrhage; hemoptysis; subconjunctival hemorrhage; hematoma >5 cm or leading to prolonged or new hospitalization; being clinically overt with a decrease of hemoglobin ≧2 to <3 g/dL; any decrease in hemoglobin ≧3 g/dL with any transfusion of at least 1 unit of packed red blood cells or whole blood; or protamine sulfate administered for uncontrolled bleeding.

The main secondary efficacy end point was the achievement of therapeutic anticoagulation at the beginning and end of the procedure. The proportion of enoxaparin patients achieving target anti-Xa levels (analyzed centrally) of 0.5 to 1.8 IU/mL was compared with the proportion of UFH patients achieving target ACT (200 to 300 seconds with GP IIb/IIIa inhibitors or 300 to 350 seconds without), at both start and end of procedure. Other secondary outcome measures were:

1. The composite end point of non-CABG-related major bleeding up to 48 hours after the index PCI, all-cause mortality, nonfatal myocardial infarction (defined as new significant Q wave in ≧2 leads; elevation of total creatine kinase or MB fraction ≧2 times the upper limit of normal, or urgent target vessel revascularization at 30 days.

2. The composite end points of all-cause mortality or nonfatal myocardial infarction, and all-cause mortality, nonfatal myocardial infarction, or urgent target vessel revascularization, whichever occurred first.

All events were adjudicated by a blinded independent clinical events committee.

Statistical Analysis

A sample size of 2700 patients was initially calculated based on an incidence of all bleeding up to 48 hours of 7.0% for the UFH group and a corresponding incidence of up to 3.7% in the enoxaparin group (47% risk reduction); the use of a type 1, two-sided error rate of 2.5% for each comparison of the enoxaparin versus UFH group, giving a global alpha level of 5%; the use of uncorrected Chi-squared tests; a power of 80%; and the assumption that approximately 2% of patients may not be treated. At a planned interim evaluation, the sample size was reevaluated and because the overall bleeding rate (3.5%) was lower than the anticipated 4.8%, a final number of 3690 patients was set. Analyses were performed on the intent-to-treat population and were adjusted for time of randomization. For bleeding and target anti-Xa and ACT analyses, adjustment was also made for the use of GP IIb/IIIa inhibitors. Results for the per protocol population were similar to the intent-to-treat population. Analyses were performed using SAS statistical software version 8.2 (SAS Institute Inc, Cary, N.C., USA). The incidence of the primary end point was compared between the enoxaparin and UFH groups using logistic regression analysis. If superiority of enoxaparin over UFH was not observed, the noninferiority of enoxaparin to UFH was tested using a two-sided, adjusted confidence interval of the differences in event rates as planned; the noninferiority margin was set at 30% of the observed bleeding rates in the UFH group.

The target anti-Xa or ACT endpoint was analyzed using a logistic regression model. The objective for the composite quadruple end point was to show noninferiority of the enoxaparin doses, using a two-sided confidence interval of the difference in event rates; the noninferiority margin was set at 39% of the observed rates in the UFH group. Time-to-event analyses for the other secondary end points up to day 30 were performed using Cox proportional hazard models. For primary and secondary analyses, each enoxaparin dose was compared with UFH. The Simes adjustment for multiplicity was applied to ensure a global type 1 error of 0.05: if both P values are ≦0.05, both are significant; if the highest P value is >0.05, the other P value has to be ≦0.025 to be significant.

An independent Data Monitoring Committee (DMC) monitored the progress of the trial and ensured that the safety of patients was not compromised. After reviewing all reported serious adverse events in 3216 randomized patients, the DMC recommended that randomization to the enoxaparin 0.5 mg/kg group be discontinued. Even though the numbers were small, data at the time suggested that the all-cause mortality in the enoxaparin 0.5 mg/kg group tended to be higher than either the UFH group or the enoxaparin 0.75 mg/kg group. However, there was no significant difference between the 0.5 mg/kg group and the UFH group. Also, there was no significant difference when the two enoxaparin groups were analyzed together and compared with the UFH group. Although the Steering Committee did not concur with the DMC recommendation a conservative approach was followed and enrollment was suspended in the enoxaparin 0.5 mg/kg group just short of full study completion.

Results

Patient characteristics

Between January 2004 and December 2004, 3528 patients were randomized to receive enoxaparin 0.5 mg/kg intravenously (1070 patients), enoxaparin 0.75 mg/kg intravenously (1228 patients), or UFH intravenously (1230 patients). Baseline characteristics were well balanced between the 3 treatment groups (Table 1).

Patient evaluability was similar between the 3 groups: 98.2% of subjects were evaluable for the primary end point, 92.1% for anti-Xa or ACT measurements, 97.0% for the composite quadruple end point, and all randomized subjects for time-to-event analysis at 30 days.

Procedure Characteristics

The three groups were comparable for all procedural characteristics (Table 1). Of patients receiving UFH, 16.5% received at least one additional bolus because of a low ACT. At least one additional bolus of enoxaparin for prolonged procedures (>2 hours) was given to 0.6% of patients receiving enoxaparin 0.5 mg/kg and 0.2% of patients receiving enoxaparin 0.75 mg/kg. The median ACT at the start and end of PCI was 336 and 292 seconds for patients receiving UFH only and 300 and 255 seconds for patients receiving UFH with GP IIb/IIIa inhibitors.

Primary End Point

Non-CABG-related major and minor bleeding at 48 hours occurred in 6.0% of patients in the enoxaparin 0.5-mg/kg group, 6.6% of patients in the enoxaparin 0.75-mg/kg group, and 8.7% of patients in the UFH group (FIG. 1). This represents a significant 31% reduction in the primary end point with enoxaparin 0.5 mg/kg compared with UFH, meeting the criteria for superiority over UFH (P=0.014). A nonsignificant trend in favor of enoxaparin 0.75 mg/kg compared with UFH (24% reduction; P=0.052) was observed, meeting the pre-specified criteria for noninferiority (95% confidence interval of the difference −4.1% to 0.0%, excluding the noninferiority margin of 2.6%). This beneficial effect was primarily driven by the reduction in non-CABG-related major bleeding, which was significantly reduced in both the enoxaparin 0.5 mg/kg group (1.2% vs. 2.8%, P=0.005) and the 0.75 mg/kg group (1.2% vs. 2.8%, P=0.007), compared with UFH (FIG. 1). There was no significant difference in the rate of minor bleeding between either enoxaparin group and UFH. Transfusion rates up to 48 hours were extremely low at 0.5%, 0.8%, and 1.0% for the enoxaparin 0.5 mg/kg, 0.75 mg/kg, and UFH groups, respectively.

Consistent results were found across all the major subgroups for the primary end point. By multivariate analysis, significant independent correlates of the primary end point included treatment with enoxaparin 0.5 mg/kg (FIG. 2).

Secondary Endpoints

Significantly more patients receiving enoxaparin achieved prespecified target anticoagulation levels (main secondary efficacy end point) compared with patients receiving ACT-adjusted UFH (Table 2).

When a narrower anti-Xa range was selected (0.5 to 1.2 IU/mL) that corresponded to adequate anticoagulation levels in the treatment of ACS, 75.5% and 59.4% of patients reached the anti-Xa target for enoxaparin 0.5 mg/kg and 0.75 mg/kg, respectively, with a significant difference compared with UFH being maintained (P<0.001 for both comparisons). At the start of PCI, median anti-Xa levels were 0.81 and 1.09 IU/mL for the enoxaparin 0.5 and 0.75 mg/kg groups, respectively; at the end of PCI these values were 0.68 and 0.93 IU/mL, respectively.

The composite quadruple end point occurred in 7.2% of patients receiving enoxaparin 0.5 mg/kg and in 7.9% of patients receiving enoxaparin 0.75 mg/kg, compared with 8.4% of patients receiving UFH, meeting the prespecified criteria for noninferiority for both enoxaparin doses (Table 2). The incidence of the all-cause mortality or nonfatal myocardial infarction at 30 days was similar between patients receiving either dose of enoxaparin or UFH (FIG. 3). The composite of all-cause mortality, nonfatal myocardial infarction, or urgent target vessel revascularization at 30 days and the individual components are reported in Table 2.

Discussion

STEEPLE is the first large, randomized controlled trial to demonstrate the superior safety of intravenous enoxaparin over unfractionated heparin in the modern era of PCI that includes the use of drug-eluting stents, clopidogrel and GP IIb/IIIa inhibitors. Enoxaparin (0.5 mg/kg) significantly reduced the primary end-point of any bleeding compared with an ACT-adjusted UFH regimen. Importantly, both enoxaparin groups showed a highly significant 57% reduction in major bleeding over UFH. In terms of efficacy, both doses of enoxaparin provided similar protection from 30-day ischemic events compared with UFH These findings translated into nonsignificant reductions with enoxaparin for the quadruple end point; non-inferiority testing was satisfied for both enoxaparin groups. The clinical benefit observed with enoxaparin came with a superior ease of use compared with UFH; enoxaparin is used as a single intravenous bolus before the start of PCI, without anticoagulation monitoring, a similar dose is used with or without GP IIb/IIIa inhibitors, and there is the possibility of immediate sheath removal after PCI with enoxaparin 0.5 mg/kg.

The heparin dosing used in the STEEPLE trial, based on ACC/AHA and ESC guidelines, resulted in among the lowest rates of bleeding complications published to date. Nevertheless, the enoxaparin strategies employed in this study resulted in even lower major and minor hemorrhagic complications compared with UFH.

Enoxaparin results in a significant 4-fold increase in rate of patients achieving target anticoagulation levels compared with UFH. This highlights the better bioavailability of enoxaparin over UFH, which requires careful and fastidious coagulation monitoring. Despite the strict monitoring imposed by the controlled nature of the trial, 80% of patients administered UFH were not in the anticoagulation target range during the time of procedure—a percentage likely to be even higher in “real-world” practice. Whether these patients who are out of target range have worse outcomes than those within the target range is uncertain. Studies have demonstrated a poor relationship between ACT and ischemic or bleeding events, thus making the value of systematic measurement of ACT values in patients on UFH questionable. In contrast, in a large unselected population of patients with unstable angina or non-ST-segment elevation myocardial infarction, it was shown that anti-Xa levels were independently associated with mortality at 30 days. However, the optimal anti-Xa level with enoxaparin for patients undergoing PCI is still unknown and will be explored in further analysis of the STEEPLE trial data. Even when aiming for a tighter anti-Xa range (0.5 to 1.2 IU/mL), enoxaparin remained significantly more predictable and stable than UFH anticoagulation: more patients reached the target with the 0.5 mg/kg enoxaparin dose.

The important safety benefit seen with enoxaparin did not come at a cost of increased numbers of ischemic events. The double end point of all-cause mortality and myocardial infarction, an indicator of hard clinical outcome, was similar between the three treatment groups. Between patients treated with enoxaparin and UFH, there was no significant difference in the triple ischemic endpoint or any individual component of the composite endpoint, including mortality. The mortality rate in the trial was low and comparable to other recent PCI trials (n=18; 0.5%). The premature termination of the low dose enoxaparin group was a conservative step in response to a request from the DMC and was based on an apparent but non-significant increase in the all-cause mortality versus the UFH group. Further evaluation of the causes of death, as well as other adverse events not captured as study end points, was not suggestive of a relationship between enoxaparin dose and ischemic events. Given that all components of the composite efficacy endpoint were lower in the enoxaparin 0.5 mg/kg group than in the other two groups (except all-cause mortality, which was a very rare event), this suggests that the non-significant trend observed for mortality is due to chance. Despite the premature discontinuation of the enoxaparin 0.5 mg/kg group, 1,070 patients were already randomized and thus represent an appropriate and well-balanced group to be compared with the other treatment groups.

According to the STEEPLE results, enoxaparin, with or without GP IIb/IIIa inhibitors, appears to be a favorable alternative to UFH in non-emergent PCI. The REPLACE-2 trial showed that bivalirudin plus GP IIb/IIIa inhibitors was noninferior to UFH plus planned GP IIb/IIIa inhibitors for the primary endpoint of death, myocardial infarction, urgent revascularization and major bleeding. However, bivalirudin is administered as a bolus followed by an intravenous infusion. In contrast, enoxaparin is administered as a single bolus.

In summary, the STEEPLE trial has confirmed that intravenous enoxaparin for the simple management of patients scheduled to undergo elective PCI is an attractive alternative to UFH anticoagulation in the catheterization laboratory.

TABLE 1
Patient and procedure characteristics.

| | Enoxaparin | | Enoxaparin | | | |
|---|---|---|---|---|---|---|
| | 0.5 mg/kg | | 0.75 mg/kg | | UFH | |
| Characteristic | (n = 1070) | | (n = 1228) | | (n = 1230) | |
| Age, years (mean ± SD) | 63.4 ± 10.5 | | 63.0 ± 10.2 | | 63.5 ± 10.2 | |
| Age ≧75 years, % | 16.7 | | 14.7 | | 15.0 | |
| Men, % | 74.7 | | 76.1 | | 74.0 | |
| Weight, kg (mean ± SD) | 84.0 ± 16.9 | | 84.2 ± 16.7 | | 83.3 ± 16.0 | |
| Creatinine clearance, % | | | | | | |
| >30 to ≦60 mL/min | 17.4 | | 18.5 | | 18.0 | |
| ≦30 mL/min | 1.3 | | 0.5 | | 1.6 | |
| Diabetes, % | 30.3 | | 29.2 | | 30.9 | |
| Prior myocardial infarction, % | | | | | | |
| >48 hours to ≦7 days | 1.5 | | 2.0 | | 2.0 | |
| ≦48 hours | 0.4 | | 0.7 | | 0.2 | |
| Prior unstable angina, % | | | | | | |
| >48 hours to ≦7 days | 6.7 | | 7.0 | | 6.7 | |
| ≦48 hours | 5.1 | | 5.9 | | 4.9 | |
| Prior PCI, % | 34.7 | | 36.5 | | 38.9 | |
| Prior CABG, % | 14.4 | | 12.7 | | 15.1 | |
| Platelet count <80,000/mm³, % | 0.1 | | 0.1 | | 0.0 | |
| Hemoglobin <10 g/dL, % | 1.7 | | 3.2 | | 3.2 | |
| Concomitant medications | | | | | | |
| Prior to PCI: | | | | | | |
| Chronic treatment with thienopyridine | 510 | (47.7) | 538 | (43.8) | 590 | (48.0) |
| Chronic treatment with aspirin | 891 | (83.3) | 1056 | (86) | 1063 | (86.4) |
| Day of PCI: | | | | | | |
| GP IIb/IIIa inhibitor used | 433 | (40.5) | 499 | (40.6) | 491 | (39.9) |
| Thienopyridine | 1007 | (94.2) | 1158 | (94.5) | 1170 | (95.1) |
| Aspirin | 986 | (92.3) | 1147 | (93.7) | 1159 | (94.4) |
| Thrombolytics | 7 | (0.7) | 7 | (0.6) | 8 | (0.7) |
| PCI variables | | | | | | |
| PCI performed | 1035 | (96.7) | 1205 | (98.1) | 1205 | (98.0) |
| Stent procedure | 974 | (94.1) | 1126 | (93.4) | 1133 | (94.0) |
| Drug eluting stent | 608 | (58.7) | 669 | (55.5) | 675 | (56.0) |
| Target vessel | | | | | | |
| LAD | 486 | (47.0) | 564 | (46.8) | 546 | (45.3) |
| LCX | 329 | (31.8) | 362 | (30.0) | 375 | (31.1) |
| RCA | 376 | (36.3) | 456 | (37.8) | 484 | (40.2) |
| LMA | 13 | (1.3) | 17 | (1.4) | 12 | (1.0) |
| Saphenous or artery graft | 43 | (4.2) | 49 | (4.1) | 43 | (3.6) |
| Multi-vessel intervention (≧2 vessels) | 161 | (15.6) | 184 | (15.3) | 207 | (17.2) |
| Closure device used | 408 | (39.5) | 470 | (39.1) | 478 | (39.7) |

| | | | |
|---|---|---|---|
| Median time from end of PCI to sheath | 0:54 | 3:14 | 2:24 |
| removal, hours:mins (IQR) | (0:01, 4:19) | (0:01, 4:55) | (0:01, 4:14) |

GP, glycoprotein;
LAD, left anterior descending;
LCX, left circumflex;
LMA, left main artery;
PCI, percutaneous coronary intervention;
RCA, right coronary artery;
SD, standard deviation;
IQR, interquartile ranges;
UFH, unfractionated heparin.

TABLE 2
Secondary end points of the STEEPLE trial.

| | Enoxaparin | | | Enoxaparin | | | |
|---|---|---|---|---|---|---|---|
| | 0.5 mg/kg | | | 0.75 mg/kg | | | UFH |
| A. End points | (n = 1070) | | | (n = 1228) | | | (n = 1230) |
| | | Difference vs. | | | Difference vs. | | |
| | n/N (%) | UFH, (95% CI) | P value* | n/N (%) | UFH, (95% CI) | P value* | n (%) |
| Composite of non-CABG- | 74/1032 (7.2) | −0.8 (−3.2, 1.5) | 0.477 | 94/1191 (7.9) | −0.5 (−2.7, 1.6) | 0.636 | 101/1198 (8.4) |
| related major bleeding up to | | | | | | | |
| 48 hours, all-cause mortality, | | | | | | | |
| nonfatal MI, or UTVR at 30 | | | | | | | |
| days^{†} | | | | | | | |
| | | Hazard ratio, | | | Hazard ratio, | | |
| | n/N (%) | (95% CI) | P value* | n/N (%) | 95% CI | P value* | n (%) |
| Composite of all-cause | 66/1070 (6.2) | 1.12 (0.79-1.60) | 0.513 | 84/1228 (6.8) | 1.18 (0.86-1.62) | 0.298 | 72/1230 (5.8) |
| mortality, nonfatal MI, or | | | | | | | |
| UTVR at 30 days^{‡} | | | | | | | |
| Composite of all-cause | 59/1070 (5.6) | 1.02 (0.71, 1.47) | 0.914 | 79/1228 (6.4) | 1.14 (0.83, 1.58) | 0.419 | 70/1230 (5.6) |
| mortality, nonfatal MI at 30 | | | | | | | |
| days^{‡} | | | | | | | |
| All-cause mortality at 30days^{‡} | 10/1070 (1.0) | 3.35 (0.92, 12.19) | 0.066 | 3/1228 (0.2) | 0.61 (0.15, 2.54) | 0.495 | 5/1230 (0.4) |
| Non-fatal MI at 30 days^{‡} | 50/1070 (4.7) | 0.91 (0.62, 1.34) | 0.636 | 76/1228 (6.1) | 1.18 (0.85, 1.65) | 0.322 | 65/1230 (5.2) |
| UTVR at 30 days^{‡} | 8/1070 (0.8) | 1.35 (0.47, 3.88) | 0.582 | 14/1228 (1.1) | 1.77 (0.74, 4.21) | 0.199 | 8/1230 (0.7) |
| Within target anticoagulation | 771/978 (78.8) | | <0.001 | 1045/1139 (91.7) | | <0.001 | 223/1134 (19.7) |
| range during the procedure^{§} | | | | | | | |

CABG, coronary artery bypass graft; MI, myocardial infarction, UFH, unfractionated heparin; UTVR, urgent target vessel revascularization.
*P values are for the comparison of enoxaparin groups with the UFH group.
^{†}Observed frequencies; 95% confidence interval for adjusted differences between groups for the composite event rate of non-CABG major bleeding up to 48 hours, all-cause mortality, non-fatal MI, or UTVR at 30 days: enoxaparin 0.5 mg/kg and UFH, (−3.2 to 1.5%), enoxaparin 0.75 mg/kg and UFH, (−2.7 to 1.6%); noninferiority margin of 3.3%.
^{‡}Reported as hazard rates
^{§}Anti-Xa levels of 0.5 to 1.8 IU/mL for enoxaparin, ACTs of 200 to 300 seconds with GP IIb/IIIa inhibitors or 300 to 350 seconds without GP IIb/IIIa inhibitors for UFH

While the administration of enoxaparin sodium has been described in connection with certain embodiments, it is not intended to limit the invention to the particular forms set forth, but on the contrary, it is intended to cover such alternatives, modifications and equivalents as may be included within the spirit and scope of the invention as defined by the following claims.

What is claimed is:

1. A method for performing percutaneous coronary intervention in a patient in need thereof comprising administering intravenously a bolus comprising an effective amount of enoxaparin to the patient after sheath insertion and prior to the percutaneous coronary intervention.

2. The method of claim 1, wherein sheath removal occurs immediately after the percutaneous coronary intervention.

3. The method of claim 1, wherein sheath removal occurs 4 to 6 hours after the percutaneous coronary intervention.

4. The method of claim 1, further comprising administering a second bolus of enoxaparin during the percutaneous coronary intervention.

5. The method of claim 4, wherein the amount of enoxaparin in the second bolus is less than the amount of enoxaparin initially administered.

6. The method of claim 5, wherein the amount of enoxaparin in the second bolus is about half of the amount of enoxaparin initially administered.

7. The method of claim 1, wherein enoxaparin is administered in an amount such that the patient achieves an anti-Xa level of 0.5 to 1.8 IU/mL

8. The method of claim 5, wherein enoxaparin is administered in an amount such that the patient achieves an anti-Xa level of 0.5 to 1.2 IU/mL.

9. The method of claim 1, wherein enoxaparin is administered in an amount sufficient to reduce the primary end-point of any bleeding as compared with an ACT-adjusted UFH regimen.

10. The method of claim 1, wherein enoxaparin is administered at a dosage of 0.5 mg/kg.

11. The method of claim 1, wherein enoxaparin is administered at a dosage of 0.75 mg/kg.

12. The method of claim 1, wherein the patient exhibits a significant reduction in major bleeding as compared with a subject who had been administered UFH prior to undergoing percutaneous coronary intervention.

13. The method of claim 12, wherein the significant reduction in major bleeding is at least 25%.

14. The method of claim 13, wherein the significant reduction in major bleeding is at least 40%.

15. The method of claim 14, wherein the significant reduction in major bleeding is at least 55%.

16. The method of claim 1, further comprising administering at least one additional therapeutic agent.

17. The method of claim 16, wherein the at least one additional therapeutic agent is chosen from aspirin and thienopyridines.

18. The method of claim 16, wherein the at least one additional therapeutic agent is chosen from clopidogrel and GP IIb/IIIa inhibitors.

19. The method of claim 18, wherein the at least one additional therapeutic agent is chosen from GP IIb/IIIa inhibitors.

20. The method of claim 19, wherein enoxaparin is administered in about the same amount as when the GP IIb/IIIa inhibitor is not administered.

21. The method of claim 1, further comprising implanting into a blood vessel of the patient a drug-eluting stent.

22. The method of claim 1, wherein the patient achieves target anticoagulation level at a rate significantly increased as compared to the rate for a subject receiving UFH.

23. The method of claim 22, wherein the patient achieves target anticoagulation level at a rate at least 2-fold faster than the rate for a subject receiving UFH.

24. The method of claim 23, wherein the patient achieves target anticoagulation level at a rate at least 3-fold faster than the rate for a subject receiving UFH.

25. The method of claim 24, wherein the patient achieves target anticoagulation level at a rate 4-fold faster than the rate for a subject receiving UFH.

26. A method for preventing thrombosis in a human patient in need thereof comprising administering intravenously a bolus comprising an effective amount of enoxaparin to the patient after sheath insertion and prior to the percutaneous coronary intervention.

27. The method of claim 26, wherein sheath removal occurs immediately after the percutaneous coronary intervention.

28. The method of claim 26, wherein sheath removal occurs 4 to 6 hours after the percutaneous coronary intervention.

29. The method of claim 26, further comprising administering a second bolus of enoxaparin during the percutaneous coronary intervention.

30. The method of claim 29, wherein the amount of enoxaparin in the second bolus is less than the amount of enoxaparin initially administered.

31. The method of claim 30, wherein the amount of enoxaparin in the second bolus is about half of the amount of enoxaparin initially administered.

32. The method of claim 26, wherein enoxaparin is administered in an amount such that the patient achieves an anti-Xa level of 0.5 to 1.8 IU/mL

33. The method of claim 32, wherein enoxaparin is administered in an amount such that the patient achieves an anti-Xa level of 0.5 to 1.2 IU/mL.

34. The method of claim 26, wherein enoxaparin is administered in an amount sufficient to reduce the primary end-point of any bleeding as compared with an ACT-adjusted UFH regimen.

35. The method of claim 26, wherein enoxaparin is administered at a dosage of 0.5 mg/kg.

36. The method of claim 26, wherein enoxaparin is administered at a dosage of 0.75 mg/kg.

37. The method of claim 26, wherein the patient exhibits a significant reduction in major bleeding as compared with a subject who had been administered UFH prior to undergoing percutaneous coronary intervention.

38. The method of claim 37, wherein the significant reduction in major bleeding is at least 25%.

39. The method of claim 38, wherein the significant reduction in major bleeding is at least 40%.

40. The method of claim 39, wherein the significant reduction in major bleeding is at least 55%.

41. The method of claim 26, further comprising administering at least one additional therapeutic agent.

42. The method of claim 41, wherein the at least one additional therapeutic agent is chosen from aspirin and thienopyridines.

43. The method of claim 41, wherein the at least one additional therapeutic agent is chosen from clopidogrel and GP IIb/IIIa inhibitors.

44. The method of claim 41, wherein the at least one additional therapeutic agent is chosen from GP IIb/IIIa inhibitors.

45. The method of claim 44, wherein enoxaparin is administered in about the same amount as when the GP IIb/IIIa inhibitor is not administered.

46. The method of claim 26, further comprising implanting into a blood vessel of the patient a drug-eluting stent.

47. The method of claim 26, wherein the patient achieves target anticoagulation level at a rate significantly increased as compared to the rate for a subject receiving UFH.

48. The method of claim 47, wherein the patient achieves target anticoagulation level at a rate at least 2-fold faster than the rate for a subject receiving UFH.

49. The method of claim 48, wherein the patient achieves target anticoagulation level at a rate at least 3-fold faster than the rate for a subject receiving UFH.

50. The method of claim 49, wherein the patient achieves target anticoagulation level at a rate 4-fold faster than the rate for a subject receiving UFH.

51. A method for treating thrombosis in a human patient in need thereof comprising administering intravenously a bolus comprising an effective amount of enoxaparin to the patient after sheath insertion and prior to the percutaneous coronary intervention.

52. The method of claim 51, wherein sheath removal occurs immediately after the percutaneous coronary intervention.

53. The method of claim 51, wherein sheath removal occurs 4 to 6 hours after the percutaneous coronary intervention.

54. The method of claim 51, further comprising administering a second bolus of enoxaparin during the percutaneous coronary intervention.

55. The method of claim 54, wherein the amount of enoxaparin in the second bolus is less than the amount of enoxaparin initially administered.

56. The method of claim 55, wherein the amount of enoxaparin in the second bolus is about half of the amount of enoxaparin initially administered.

57. The method of claim 51, wherein enoxaparin is administered in an amount such that the patient achieves an anti-Xa level of 0.5 to 1.8 IU/mL

58. The method of claim 57, wherein enoxaparin is administered in an amount such that the patient achieves an anti-Xa level of 0.5 to 1.2 IU/mL.

59. The method of claim 51, wherein enoxaparin is administered in an amount sufficient to reduce the primary end-point of any bleeding as compared with an ACT-adjusted UFH regimen.

60. The method of claim 51, wherein enoxaparin is administered at a dosage of 0.5 mg/kg.

61. The method of claim 51, wherein enoxaparin is administered at a dosage of 0.75 mg/kg.

62. The method of claim 51, wherein the patient exhibits a significant reduction in major bleeding as compared with a subject who had been administered UFH prior to undergoing percutaneous coronary intervention.

63. The method of claim 62, wherein the significant reduction in major bleeding is at least 25%.

64. The method of claim 63, wherein the significant reduction in major bleeding is at least 40%.

65. The method of claim 64, wherein the significant reduction in major bleeding is at least 55%.

66. The method of claim 51, further comprising administering at least one additional therapeutic agent.

67. The method of claim 66, wherein the at least one additional therapeutic agent is chosen from aspirin and thienopyridines.

68. The method of claim 66, wherein the at least one additional therapeutic agent is chosen from clopidogrel and GP IIb/IIIa inhibitors.

69. The method of claim 68, wherein the at least one additional therapeutic agent is chosen from GP IIb/IIIa inhibitors.

70. The method of claim 69, wherein enoxaparin is administered in about the same amount as when the GP IIb/IIIa inhibitor is not administered.

71. The method of claim 51, further comprising implanting into a blood vessel of the patient a drug-eluting stent.

72. The method of claim 51, wherein the patient achieves target anticoagulation level at a rate significantly increased as compared to the rate for a subject receiving UFH.

73. The method of claim 72, wherein the patient achieves target anticoagulation level at a rate at least 2-fold faster than the rate for a subject receiving UFH.

74. The method of claim 73, wherein the patient achieves target anticoagulation level at a rate at least 3-fold faster than the rate for a subject receiving UFH.

75. The method of claim 74, wherein the patient achieves target anticoagulation level at a rate 4-fold faster than the rate for a subject receiving UFH.
